(19) United States
(12) Patent Application Publication
(10) Pub. No.: US 2009/0023166 A1
(43) Pub. Date: Jan. 22, 2009

(54) Method For the in Vitro Diagnosis of Autoimmune Immune Response by Detection of Antibodies Directed Against The Pentraxin 3 Antigen

(76) Inventors: Pascale Jeannin, Bouchemaine (FR); Yves Delneste, Bouchemaine (FR); Alberto Mantovani, Milan (IT)

Correspondence Address: PAULEY PETERSEN & ERICKSON, 2800 WEST HIGGINS ROAD, SUITE 365, HOFFMAN ESTATES, IL, 60195, US

(21) Appl. No.: 12/223,004

(22) PCT Filed: Jan. 19, 2007
(86) PCT No.: PCT/EP2007/050562

§ 371 Date: Jul. 18, 2008

(30) Foreign Application Priority Data
Jan. 20, 2006 (FR) 06/00516

(51) Int. Cl. G01N 33/53; C12Q 1/00; G01N 33/566

(52) U.S. Cl. 435/7.92; 435/7.1

(57) ABSTRACT

A method for the in vitro diagnosis of an autoimmune immune response in an individual by detection, in a biological fluid from said individual, of antibodies directed against the pentraxin 3 (PTX3) antigen, characterized in that the presence of antibodies directed against the PTX3 antigen (anti-PTX3 antibodies) are determined in a biological fluid from the individual, and kits for implementing the method.

The invention relates to a method for in vitro diagnosis of an autoimmune immune response in a subject by detection in the biological fluid of said subject of antibodies directed against the pentraxin 3 (PTX3) antigen characterised in that the presence of antibodies directed against the PTX3 antigen (anti-PTX3 antibodies) is determined. It also relates to kits for implementing this method.

### I. STATE OF THE ART

### 1. Lymphocytes in the Immune Response

a. Lymphocytes and the Anti-Self Physiological Immune Response

The role of cells of the immune system, particularly lymphocytes (B and T), is the destruction of foreign agents such as microorganisms in the body. Lymphocytes express receptors which recognise microbial agents. Thus for example, T lymphocytes express the T receptor and B lymphocytes express immunoglobulins, also called antibodies. When activated, lymphocytes specific for microorganism constituents release mediators which destroy microorganisms and/or cells infected by microorganisms.

Broadly speaking, these mediators are immunoglobulins (or antibodies) in the case of B lymphocytes. These antibodies bind to microorganisms and/or to infected cells and stimulate their elimination by other immune cells such as macrophages, polynuclear or natural killer cells, also called NK cells. The mediators that facilitate the elimination of microorganisms producted by T lymphocytes are cytokines and/or toxic mediators.

Thus, recognition of a microorganism (considered to be non-self) by specific lymphocytes generally leads to its destruction by cells of the immune system.

b. Autoimmune Processes and Autoimmune Diseases

In addition to B and T lymphocytes which carry receptors that recognise non-self constituents such as microorganisms, all subjects also carry lymphocytes which recognise self-constituents. Under normal physiological conditions, these lymphocytes are not activated. They are maintained by means of various mechanisms in a non-reactive state to the self, called tolerance or anergy.

Under certain ill-defined conditions, for example viral infection or genetic predisposition, these lymphocytes may be activated. This then results in the development of an abnormal and damaging immune response leading to tissue destructions. The immune system, and in particular lymphocytes, act as if cells and/or certain molecules of the subject were foreign and mobilize their whole arsenal to destroy these cells. Consequently, activated lymphocytes specific to the self then react by releasing toxic antibodies and/or mediators.

This lymphocytes reaction against self-constituents is what characterises the autoimmune processes. If this reaction results in the development of a pathology, it is called an autoimmune disease. Nevertheless, an autoimmune type disorder can also be found associated with many other pathologies, such as chronic inflammation or even pathologies associated with considerable cell lysis.

### 2. Autoantibodies

a. Definitions

In the case of an autoimmune type immunological disorder, B lymphocytes specific of the self are activated and produce immunoglobulins that are specific to these constituents. These antibodies are called autoantibodies. Self molecules and/or structures recognised by antibodies are called autoantigens. Self-constituents are expressed by tissues, cells or produced by cells in all subjects. Some of these molecules are structural proteins while others play a well-established role, for example molecules involved in coagulation and/or in destruction/elimination of bacterial constituents.

b. Target Antigens of Autoantibodies.

Currently, a hundred or so of molecules have been identified as being autoantibody targets. Examples of these include:

anti-phospholipid antibodies directed against two plasma proteins bound to anionic phospholipids: β2-glycoprotein I and prothrombin,

soluble anti-nuclear antibodies directed against antigens Sm, Sc170, SSA, SSB, and Jol.,

anti-polynuclear neutrophil antibodies directed against proteins such as myeloperoxidase, proteinase 3 and, less frequently, against the BIP molecule (bactericidal increasing protein), azurocidin, elastase and cathepsin G.

c. Physiopathological Role of Autoantibodies.

In addition to a central role in the diagnosis of immune disorders, some autoantibodies play an active role in the pathogenicity of the disease. For example, circulating immune complexes deposits (autoantibodies/autoantigen complexes) become deposited in kidneys and cause glomerulonephritis (acute inflammation of kidney). Anti-nucleosome antibodies produced in the course of disseminated lupus erythematosis (DLE) are also directly involved in the production of lesions in kidneys. In some special cases, the presence of autoantibodies changes the function of the autoantigen and has physiopathological consequences.

Nonetheless, at present, while a link between the presence and level of autoantibodies and the existence of organ disorders is suspected, the physiopathological role is not known for the majority of autoantibodies of known specificity.

d. Immunological Diagnosis Allowing Investigation of Autoantibodies.

At present, the most commonly used techniques in biology and immunology laboratories to detect an autoimmune process consist in investigating the presence of autoantibodies in patient sera.

The presence of autoantibodies capable of binding to various tissues, whether human or not, or to cells or proteins is analysed. Non-human tissues are sometimes used when it has been previously demonstrated that the autoantibody target is the same across species.

Techniques used to investigate autoantibodies when the autoantigen source used is tissues or cells are immuno-histochemistry and immuno-cytochemistry techniques respectively: tissue sections or cells are contacted with different dilutions of the serum being tested. After incubation then washing, immunoglobulins which recognise tissues or cells are detected using a human anti-immunoglobulin antibody coupled to a molecule which allows detection such as a fluorochrome or an enzyme-substrate complex.

When the molecules recognised by the autoantibodies are known, other techniques are used: ELISA, Immuno-Dot and/or immunoprints. In the first two cases, the molecule (autoantigen) is adsorbed on a polystyrene plate or a membrane respectively. The ELISA plate or the membrane is incubated with serum and the antibodies present in the serum which bind to the target are detected by means of a human anti-immunoglobulin antibody coupled to a molecule which allows detection such as a fluorochrome or an enzyme-substrate complex. In the case of immunoprints, a total protein extract of a cell or purified molecule migrates in polyacrylamide gel. The molecules thus separated according to their molecular weights are then transferred onto a membrane which undergoes the same process as described above.

e. Diagnostic and Prognostic Benefits of Autoantibody Detection.

The benefits of autoantibody detection reside in the indication of dysimmunity, or in other words of an abnormal immune response allowed by dysimmunity. Identification of autoantibodies is of no diagnostic benefit unless it is coupled to clinical information. In fact, the production of autoantibodies can precede any clinical symptoms. The presence of autoantibodies directed against certain autoantigens is mainly associated with certain so-called immune diseases such as anti-nuclear autoantibodies and DLE. Biological results are generally essential to complement the clinical picture in order to facilitate the therapeutic approach to be used, especially when this picture is atypical, which is often the case when the disease is detected at an early stage. Current treatments such as immunosuppressants and corticosteroids are effective but aggressive. The presence of autoantibodies, their specificity and level is therefore generally essential to assist the clinician in evaluating the risk/benefit ratio of treatments.

f. Current Benefit of Research of New Autoantibody Targets.

At present, research of new autoantibody targets appears to be a choice niche in the field of biotechnology. Autoimmune diseases are the third highest cause of morbidity in industrialised countries. Population ageing contributes to this increase. Research of new targets allows rapid and early diagnosis of an autoimmune immune response as well as completing the panel of autoantigen targets used in laboratories. There is currently a real interest in developing further diagnostic tests in addition to existing tests to benefit from earlier and more appropriate information in order to follow the course of the disease. In fact, early treatment makes it possible to avoid complications and to limit therapeutic costs of such chronic diseases. It should be noted that rapid diagnostic techniques also seem to be essential when the vital prognosis is at risk, for example in the case of kidney or lung disorders.

This is precisely the object of the present invention.

### II. THE INVENTION

Indeed, the inventors have succeeded in finding a means to respond to this prospect by showing, for the first time, the presence of autoantibodies directed against the pentraxin 3 molecule (PTX3), also called TNF-inducible gene 14 (TSG-14) in the biological fluids of subjects with an autoimmune immune response.

As a result, the present invention relates to a method for in vitro diagnosis of an autoimmune immune response in a subject by detection in a biological fluid of said subject of antibodies directed against the pentraxin 3 antigen (PTX3).

### I. Pentraxin 3, a Mediator of Innate Immunity.

a. Innate Immune Receptors.

Innate immunity cells are involved in the rapid recognition of microbes. Their activation controls the propagation of microbes, in particular via the production of microbiocidal mediators as well as the development of a specific immune response. In order to avoid recognition by the innate immunity cells they encounter, microbes produce many mutations in order to enhance the heterogeneity of their constituents. In order to counteract this recognition-avoidance strategy, innate immunity cells have selected receptors capable of recognising structures that are highly preserved in microorganisms and necessary for the physiology of microorganisms. These preserved structures such as lipopolysaccharides, double stranded RNA and CpG sequences are grouped together under the term “pathogen-associated molecular pattern” or PAMPs. By definition, PAMPs are distinct from the self, shared by large groups of pathogens and are essential to their survival. They constitute true molecular signatures of microbes and their recognition triggers the antimicrobial immune response.

Receptors involved in the recognition of PAMPs are called innate immune receptors or Pattern-Recognition Receptors (PRRs). PRRs recognise a broad spectrum of microbial constituents such as sugars, proteins, lipids and nucleic acids (Medzhitov and Janeway, 2000; Janeway and Medzhitov, 2002). PRRs are expressed by innate immune cells either at the intracellular or membrane level or in the extracellular environment.

PRRs are distinguished depending on their biological function:

recognition PRRs involved in the detection and/or internalisation of microorganisms by innate immune cells, and

signalling PRRs involved in the activation of immune cells by microorganisms.

Membrane recognition PRRs belong, amongst others, to the family of purging receptors, mannose receptors, type C lectins and integrins. Soluble recognition PRRs, also called opsonins, belong to the collectins (van de Wetering et al, 2004), ficollins (Matsushita & Fujita, 2002) and pentraxins (Garlanda et al, 2005) family. The role of soluble recognition PRRs is to recognise, bind to and then favour the elimination of microbes by phagocytic cells.

The pentraxin (PTX) superfamily includes molecules that have been highly conserved in the course of evolution characterised by the presence of a “pentraxin” domain in the C-terminal region. It includes many members such as:

C-reactive protein (CRP, also called PTX1) and serum amyloid P (also called PTX2). CRP and SAP are acute phase proteins produced by liver in response to proinflammatory stimuli such as IL-6. CRP and SAP bind to a wide variety of molecules including self (complement component Clq) (Agrawal & Volanakis, 1994), modified self (apoptotic cells) (Gershov et al, 2000 Bijl et al, 2003) and non-self cells (bacteria and viruses) (Hind et al, 1984). However, the role of CRP and SAP remains poorly understood.

the PTX3 molecule, also called TSG-14 (TNF-stimulated gene 14) has been identified as a molecule whose synthesis is greatly enhanced in fibroblasts and endothelial cells in response to stimulation by TNFα or IL-1 (Breviario et al, 1992; Lee et al, 1993). PTX3 is the prototype for long pentraxins: the C-terminal area of PTX3 is homologous (17% identity) to whole CRP and contains a complementary N-terminal domain which is not homologous with any other molecule (Garlanda et al, 2005).

b. The PTX3 Molecule

The PTX3 molecule is produced by many cell types such as endothelial cells, fibroblasts, chondrocytes, myocytes, mononuclear phagocytes, dendritic cells and epithelial cells in response to a pro-inflammatory stimulus or to a microbial constituent. (Breviario et al, 1992; Abderrahim-Ferkoune et al, 2003; Alles et al, 1994; Doni et al, 2003; Goodman et al, 2000; Vouret-Craviari et al, 1997; Nauta et al, 2005; Garlanda et al, 2005). The PTX3 molecule is produced in the form of a multimer consisting of 10 to 20 sub-units (Bottazzi et al, 1997).

Contrary to CRP and SAP, many functions have been described for the PTX3 molecule:

PTX3 binds to the Clq component of complement. This binding can either activate or inhibit complement classical pathway depending on the soluble or immobilised structure of Clq (Nauta et al, 2003),

PTX3 binds to apoptotic cells and favours their elimination by phagocytic cells (Rovere et al, 2000),

PTX3 binds to certain pathogens such as Salmonella typhimurium, Pseudomonas aeruginosa and Aspergillus fumigatus, thus facilitating their ingestion by phagocytic cells (Garlanda et al, 2002; Diniz et al, 2004).

Transgenic mice which overexpress PTX3 show increased survival in an endotoxemic model (Dias et al, 2001). In contrast, PTX3-deficient mice show increased sensitivity to pulmonary aspergillosis (Garlanda et al, 2002). This set of data shows that the PTX3 molecule acts like a soluble PRR and plays a crucial role in the recognition of modified self and non-self structures.

### II. Description of the Invention

A first aspect of the present invention relates to a method for in vitro diagnosis of an autoimmune immune response in a subject by detection in a biological fluid of said subject of autoantibodies directed against the pentraxin 3 antigen (PTX3) characterised in that the presence of antibodies directed against the PTX3 antigen (anti-PTX3 antibodies) is determined in biological fluids of said subject and the existence of an autoimmune immune response in the subject is concluded on this basis.

By autoimmune immune response, it is meant the existence of B lymphocytes producing antibodies directed against self-molecules.

By “anti-PTX3 antibodies”, it is meant according to the present invention any molecule containing a “paratope” capable of binding specifically to the PTX3 protein. By “anti-PTX3 antibodiesa is also meant, according to the present invention, a homogeneous population of molecules which all contain the same “paratope” capable of binding specifically to the PTX3 protein.

The term “paratope” means the antigenic combination site contained in the Fab fragment of an antibody which is located in hypervariable or CDR regions of the V_{H} and V_{L} variable domains of an immunoglobulin heavy chain and light chain.

According to the present invention, the quantity of antibodies directed against the PTX3 antigen is determined in a biological fluid of the subject and the existence of an autoimmune immune response in the subject is established through comparison with the quantity of antibodies directed against the reference PTX3 antigen in the serum of healthy subjects.

Preferably, according to the present invention, the presence and/or quantity of antibodies directed against PTX3 is determined by detection of binding between the PTX3 antigen and the anti-PTX3 antibody.

The antigen-antibody binding reaction results from the interaction between antigen epitopes and antibody paratopes. This involves four types of non-covalent bonds (hydrogen bonds, electrostatic bonds, hydrophobic bonds and Van der Waals forces).

According to the present invention, detection of binding between the PTX3 antigen and the anti-PTX3 antibody is preferably carried out by immobilisation of the PTX3 antigen on a solid support, by precipitation reactions in liquid media and/or immunoprecipitation and/or by a gel precipitation reaction.

By precipitation reaction in liquid media, it is meant according to the present invention a reaction which consists in distributing equal quantities of a PTX3 antigen solution with increasing dilutions of a biological fluid, preferably an immune serum.

The equivalence zone (which is the point where the graph reaches its maximum) corresponds to the formation of an antigen-antibody binding.

The binding of a PTX3 antigen according to the invention to a solid support can be carried out using techniques well known to the person skilled in the art. The support can be in various forms, including bands or particles such as beads. The support surface can be polyfunctional or able to be polyfunctionalised in such a way as to bind the PTX3 antigen via covalent or non-covalent interactions which can be specific or non-specific.

For the purpose of illustration, the support on which the PTX3 antigen is immobilised can be a porous or non-porous material which is insoluble in water. The support can be hydrophilic or able to be made hydrophilic and can include inorganic powders such as silica, magnesium sulphate and aluminium; natural polymer materials, particularly cellulose and materials derived from cellulose; natural or synthetic polymers such as nitrocellulose, cellulose acetate, polyvinylchloride, polyacrylamide, reticulated dextran, agarose, polyacrylate, polyethylene, polypropylene, poly(4-methylbutene), polystyrene, polymethacrylate, polyethylene terephtalate, nylon, polyvinylbutyrite, certain types of glass such as Bioglass or ceramics.

The solid support for immobilisation of the PTX3 antigen is preferably of plastic or polypropylene type for ELISA or RIA assays, of membrane type for Western blot or Dot blot assays, of beads type or of foam type.

The term ELISA (Enzyme Linked ImmunoSorbent Assay) according to the present invention means an immunological test aimed at detecting and/or assaying anti-PTX3 antibodies in a biological fluid by immunoenzyme labelling.

The term RIA (Radioimmunology assay) according to the present invention means a radio-immunological test aimed at detecting and/or assaying anti-PTX3 antibodies in a biological fluid, based on the same principle as ELISA but using radio-immunology labelling.

The term fluorimetric assay according to the present invention designates the counting of particles, for example beads, which carry the PTX3 antigen on their surface and to which anti-PTX3 antibodies that may be present in a sample to be tested will have bound. The anti-PTX3 antibodies are detected by means of human anti-immunoglobulin antibodies coupled to a fluorochrome. Measurement of the quantity of anti-PTX3 antibodies is based on the intensity of fluorescence. This is carried out using a fluorescence analyser or cell analyser: flux cytometer or FACS (fluorescence activated cell sorter).

The term Western blot or immunotranfer according to the present invention means a technique by means of which the PTX3 antigen is separated by polyacrylamide gel electrophoresis then electrophoretically transferred onto a membrane (nitrocellulose for example). The deposition on the membrane of the biological fluid sample to be tested then makes it possible to bind anti-PTX3 antibodies that might be present in the sample to the PTX3 antigen. This is followed by detection by a second antibody labelled by an isotope, a fluorochrome or an enzyme.

The term Dot blot according to the present invention means an absorption technique which consists in depositing proteins on a nitrocellulose membrane in the form of dots then carrying out the usual immunoblot steps.

In addition, the presence and/or quantity of anti-PTX3 antibodies is determined according to the present invention by using a chromogenic substrate, by chemoluminescence, by fluorescence or by radio-labelling.

The term chromogenic substrate means the chromogenic substrate of an enzyme which eventually results in a coloured reaction detected by means of a spectrophotometer. For example, this can include X-gal/OPTG and salmon-gluc/O-Me-b-Gluc, OPD, ABTS.

The term chemoluminescence means a chemical reaction accompanied by light emission. Measurement of the light emitted makes it possible to quantify one of the reagents if the quantity of the other reagent is known. For example, this can include oxido-reduction of luminal (3-aminophthalhydrazide) with oxygenated water, for example, or any hydroxide.

The term fluorescence means the use of any fluorescent molecule such as the molecules described by ICHINOSE et al. (1991) or fluorescent derivatives of isothiocyanate, phycoerithrine, rhodamine isothiocyanate, dansyle chloride or compound XRITC, protein GFP (Green Fluorescent Protein) from the fish Aequorea Victoria and its many derivatives, or even protein YFP (Yellow Fluorescent Protein) as well as the protein luciferase.

The term radio-labelling refers to labelling with a radioactive substance. The radioactive substance can be labelled for example with an isotope chosen from among [³H], [³²P] and [¹²⁵I].

According to the present invention, ELISA or RIA assays make it possible to quantify the amount of anti-PTX3 antibodies and the Dot blot or Western blot type assays make it possible to detect higher quantities of anti-PTX3 antibodies compared to reference anti-PTX3 antibodies.

By “reference anti-PTX3 antibody quantity”, it is meant according to the present invention the quantity of anti-PTX3 antibodies obtained from the serum of a healthy subject, from a set of sera from healthy subjects or defined in an arbitrary manner.

By “healthy subject”, it is meant according to the present invention a subject without an autoimmune immune response.

The expression “anti-PTX3 antibody quantity defined in an arbitrary manner” according to the present invention means any method which allows reproduction of the mean value obtained with a pool of healthy subjects.

In an even more preferred manner, the gel precipitation reaction is chosen from among radial immunodiffusion, Ouchterlony double immunodiffusion, immunoelectrophoresis and fused rocket electrophoresis type reactions.

Bym radial immunodiffusion, also called the Mancini technique, is meant according to the present invention a gel precipitation reaction which consists in incorporating a PTX3 antigen solution in gelose and depositing a biological fluid likely to contain anti-PTX3 antibodies in wells. At equilibrium, a precipitation ring forms whose square diameter is proportional to the concentration of anti-PTX3 antibodies. Concentration is expressed by reference to a standard curve with anti-PTX3 antibodies of known concentration.

Ouchterlony double immunodiffusion according to the present invention means a gel precipitation reaction carried out as follows:

PTX3 antigen and anti-PTX3 antibody solutions are deposited in wells at a distance from each other in agarose gel. Molecules diffuse into the gel as a function of their size and form precipitation lines for each antigen and antibody system.

Each precipitation line corresponds to the respective equivalence zone, in other words to formation of an antigen-antibody binding.

This method makes it possible to carry out analysis of a biological fluid and identification of anti-PTX3 antibodies.

According to the present invention, immunoelectrophoresis means a gel precipitation reaction which involves separation of proteins by electrophoresis in agarose gel, followed by double diffusion against specific antibodies in a direction that is perpendicular to the axis of electrophoretic migration. Each equivalence zone corresponds to an antigen-antibody precipitate which results in a precipitation arc. Immunoelectrophoresis makes it possible to characterise or identify anti-PTX3 antibodies but is not a quantitative method.

According to the present invention, fused rocket electrophoresis means a gel precipitation reaction in which the PTX3 antigen incorporated into agarose gel is immobile (as a result of gel pH) and the negatively charged anti-PTX3 antibody migrates under the effect of an electric field.

The resulting precipitation arc is in a fused rocket form whose height is proportional to the concentration of anti-PTX3 antibodies.

According to the present invention, biological fluid means blood, serum, plasma, lymph, urine, saliva, cerebrospinal fluid, preferably serum.

In a preferred embodiment of the invention, the diagnostic method according to the invention is an ELISA assay and comprises the following steps:

a) Incubating serum with PTX3 antigens fixed on a solid support,

b) Washing the serum antibodies not fixed to PTX3 antigens of the solid support.

c) Adding anti-immunoglobulin antibodies coupled to a marker, said anti-immunoglobulin antibodies being capable of recognising serum antibodies.

d) Washing the anti-immunoglobulin antibodies not fixed to the solid support.

e) Detecting and/or quantifying the marker bound to the solid support and correlating it to the presence and/or quantity of serum antibodies.

The concentration is variable and is to be determined as a function of the support and PTX3 source.

The PTX3 antigens according to the present invention are chosen from whole PTX3 of human, animal or synthetic origin, one or more PTX3 fragments of human, animal or synthetic origin, and PTX3 homologue molecules, preferentially chosen from the pentraxins family and/or presenting substantial homology with the primary, secondary or tertiary sequences.

The PTX3 antigens according to the present invention are obtained from a prokaryotic recombinant system, for example in an Escherichia coli strain, a eukaryotic recombinant system, preferably CHO cells and line N50, from purification using human or animal cells, tissues or biological fluids, or by chemical synthesis, preferably peptide synthesis.

According to the present invention, peptide synthesis means solid phase synthesis of PTX3 peptides.

The anti-immunoglobulin antibodies according to the present invention are selected from the group comprising anti-immunoglobulin G, anti-immunoglobulin A, anti-immunoglobulin M, anti-immunoglobulin D, and anti-immunoglobulin E.

In this preferred embodiment of the invention, the solid support consists of microbeads or microtitre plates, such as ELISA plates, and the marker is selected from the group comprising fluorescent, chemoluminescent, enzymatic and radioactive markers.

More particularly, in this preferred embodiment of the invention, the solid support is an ELISA plate and consists of the following steps:

a) Incubating the serum with PTX3 antigens fixed to the ELISA plate

b) Washing the serum antibodies not fixed to PTX3 antigens of the ELISA plate.

c) Adding anti-immunoglobulin antibodies coupled to an enzyme, said anti-immunoglobulin antibodies being capable of recognising serum antibodies.

d) Washing the anti-immunoglobulin antibodies not fixed to the ELISA plate.

e) Adding the soluble substrate corresponding to the enzyme.

f) Reading the absorbance values of wells of the ELISA plate in an ELISA reader at an appropriate wavelength and correlating it to the presence and/or quantity of antibodies in the serum.

In addition, corresponding enzymes and soluble substrates according to this preferred embodiment of the invention are selected from the group comprising:

Alkaline phosphatase and soluble substrate 4-NitroPhenyl Phosphate (PNPP)

Peroxidase and soluble substrate orthophenylene diamine (OPD)

β-galactosidase and soluble substrate 2-nitrophenyl β-galactoside (ONPG)

Glucose 6-phosphate dehydrogenase and soluble substrate glucose-6-phosphate (G6P).

Biotin and soluble substrate streptavidin coupled to peroxidase and substrate ABTS or OPD of peroxidase.

According to the present invention, the subject is preferably a mammal, still more preferably a human being.

According to the present invention, the anti-immunoglobulin antibodies are preferentially anti-human immunoglobulin antibodies.

According to the present invention, the diagnosis of an autoimmune immune response can be combined with the existence or prediction of an autoimmune disease in the subject. In a preferred manner, the autoimmune diseases are selected from the group comprising Gougerot-Sjögren syndrome, type 1 diabetes, monoclonal gammapathy, Wegener's granulomatosis, disseminated lupus erythematosus, atheromatous disease, Crohn's disease, Horton's disease, Reiter's disease (conjunctivo-uretro-synovial syndrome), rheumatoid arthritis, haemorrhagic recto-colitis, psoriatic rheumatism, sarcoidosis, sclerodermy, multiple sclerosis and autoimmune bullous dermatoses, Basedow's disease (hyperthyroidism), Hashimoto's chronic thyroiditis (hypothyroidism), Goodpasture's syndrome, pemphigus, myasthenia, insulin resistant diabetes, autoimmune haemolytic anaemia, autoimmune thrombocytopenic purpura, polymyositis and dermatomyositis, Biermer's anaemia, glomerulonephritis, certain sterile diseases, periarteritis nodosum and Churg-Strauss syndrome, Still's disease, atrophying polychondritis, Behçet's disease and spondylarthritis.

The method for diagnosis of an autoimmune immune response associated with the existence of an autoimmune disease in a subject according to one aspect of the present invention can also include detection and/or quantification in the serum of said subject of antibodies directed against other autoantigens and preferentially the myeloperoxidase antigen (MPO) and/or proteinase 3 antigen (PR3) and/or elastase and/or BPI and/or cathepsin G and/or nuclear antigens.

According to the present invention, diagnosis of an autoimmune immune response can also be associated with a pathology characterised by tissue damage in the subject. In this case, and in a preferred manner, tissue damage in the subject is due to necrosis, particularly infarction, chronic inflammation or chronic infection.

Tissue damage is assessed by measuring the concentration in various biological fluids of molecules released when cells are damaged (these molecules can be enzymes such as transaminases or inflammatory proteins)

A second aspect of the present invention relates to a diagnostic kit for detection and/or quantification in a biological fluid of antibodies directed against the PTX3 antigen, comprising:

a) a solid support wherein the PTX3 antigen is fixed to the solid support.

The characteristics of the kit (solid support, PTX3 antigen, etc) according to the present invention are as defined above for the diagnostic method.

According to the present invention, biological fluid means blood, serum, plasma, lymph, urine, saliva, cerebro-spinal fluid, preferentially serum.

Optionally, the kit according to the present invention comprises a solution containing one or more saturation proteins which saturate the reactive sites of the solid support.

Optionally, the kit according to the present invention further comprises:

b) a solution containing anti-immunoglobulin antibodies conjugated to a marker.

According to the present invention, the anti-immunoglobulin antibodies used in the kit are preferably selected from the group comprising anti-immunoglobulin G, anti-immunoglobulin A, anti-immunoglobulin M, anti-immunoglobulin D, and anti-immunoglobulin E.

Optionally, the kit according to the present invention further comprises:

c) a washing solution.

According to the present invention, a washing solution means a buffered saline solution containing a low concentration of a detergent and/or a saturation protein, preferentially of the bovine serum albumin or gelatin type.

A “saturation protein” means a protein which saturates the reactive site of the solid support.

Preferably, the solid support used in the kit according to the present invention is an ELISA plate and the marker is an enzyme.

Optionally, the kit according to the present invention further comprises:

d) a solution containing the soluble substrate corresponding to the enzyme.

A third aspect of the present invention relates to the use of a PTX3 antigen for diagnosis of an autoimmune disease in humans.

Preferably, an ELISA test is used for the diagnosis.

A fourth aspect of the present invention relates to the use of the diagnostic method or kit according to the present invention to identify, prior to the appearance of clinical symptoms, subjects at risk from an autoimmune immune response.

Preferably, the diagnostic method or kit according to the present invention is used to monitor the evolution of an autoimmune immune response, predict progression of the disease and/or monitor the efficacy of treatment.

The term “progression of the disease” means aggravation of clinical signs.

### LEGENDS OF FIGURES

FIG. 1: Detection of anti-PTX3 antibodies in subjects presenting positive indirect immunofluorescence (IFI) associated with anti-MPO autoantibodies or anti-PR3 autoantibodies

FIG. 2: detection of anti-PTX3 antibodies in subjects with positive IFI in the absence of anti-MPO autoantibodies or anti-PR3 autoantibodies.

### EXAMPLES

### 1. Use of a Diagnostic Test Allowing Investigation of Anti-PTX3 Autoantibodies in Patient Sera

a. Description of the ELISA Test Allowing Investigation of Anti-PTX3 Antibodies

96-well ELISA plates (Maxisorb®; Nunc, Roskilde, Danemark) are incubated or not overnight at 4° C. with 100 μL of PTX3 at 10 μg/mL in carbonate/bicarbonate buffer pH=9.6. The wells are then emptied and incubated for 1 hour 30 min with 300 μL of a bovine serum albumin (BSA) solution at 1% in phosphate saline buffer 10 mM, pH=7.4. Sera from patients and healthy subjects are diluted to 1/400 in PBS buffer containing 0.5% BSA (w/v) and 0.05% Tween 20 (w/v). 100 μL of this dilution is deposited for each of the sera in a well coated with PTX3 and in an uncoated well (which allows determination of background noise for each serum sample). After incubation for 2 h at 37° C., the plates are washed 4 times with 200 μL of PBS containing 1% of Tween 20 (w/v) then incubated for 1 h30 at 37° C. with 100 μL of human anti-immunoglobulin antibody (G, A and M) coupled to biotin (Jackson ImmunoResearch, West Grove, Pa.). After 4 washings with 200 μL of PBS containing 1% of Tween 20 (w/v), 100 μL of a streptavidin solution coupled to peroxidase is incubated for 1 h at 37° C. (commercial solution diluted to 1/1000, BD Pharmingen, San Jose, Calif.). After 4 washings in PBS containing 1% Tween 20 (w/v), fixed antibodies are detected using freshly prepared ABTS substrate (Sigma, St Louis, Calif.). ELISA plates are read using a fluoreometer (λ=405 nm with a reference at λ=620 nm). The results are expressed in optical density units (OD).

An example of raw results obtained after reading is presented below. The reproducibility of results was verified by carrying out successive manipulations.

In general, results were analysed as follows:

For each ELISA plate, 20 to 30 serum samples from healthy subjects were tested in parallel to 20 to 30 samples from patients.

For each serum, the OD value obtained for the uncoated well (background noise BSA) was subtracted from the OD value obtained with wells coated with the PTX3 molecule in order to obtain a specific OD.

The specific OD values obtained with sera from healthy subjects were added together in order to establish the mean and standard deviation. A threshold value defined as the mean plus 2 standard deviations is calculated for each ELISA plate (mean±2s.d.).

The specific OD values for patient sera are considered to be positive when the OD value is greater than the mean threshold value+2sd.

In the following examples, anti-PTX3 antibodies were investigated in patients presenting anti-neutrophil autoantibodies (IFI+, corresponding to ANCA+). 3 populations were analysed:

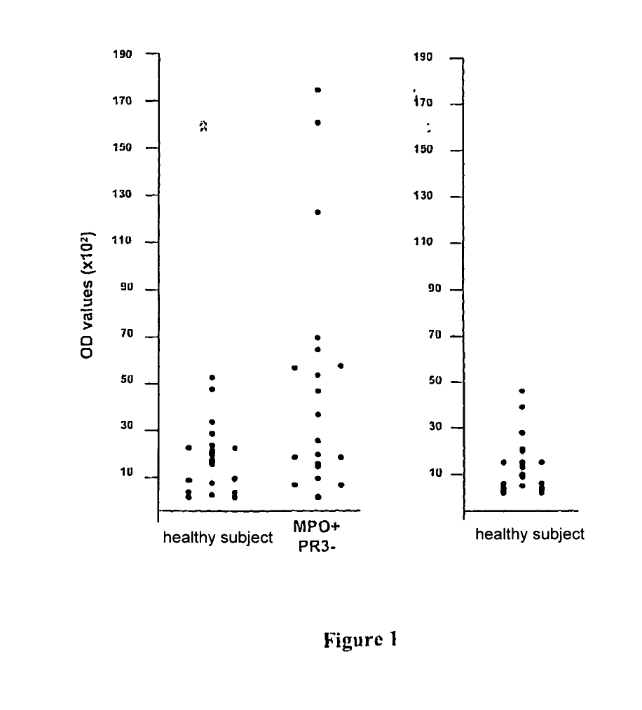

ANCA+, MPO+(PR3−) patients, example a and FIG. 1, right-hand diagram.

ANCA+PR3+(MPO−) patients, example b and FIG. 1, left hand diagram

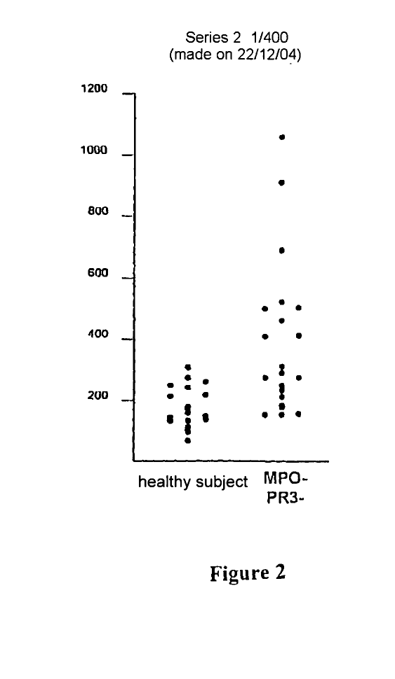

ANCA+PR3−MPO− patients, example c and FIG. 2.

example d: among the PR3− and MPO− patients, we re-analysed the preceding results (example c) as a function of the presence of autoantibodies directed against other specificities

presenting various autoantibodies.

b. Presence of Anti-PTX3 Antibodies in IFI+MPO+(PR3−) Patients

Sera from 22 patients with anti-polynuclear autoantibodies (IFI+) and anti-MPO antibodies and sera from 23 healthy subjects were tested as described above. The threshold value (mean+2sd) is 0.037-9 patient sera were higher than the threshold value. The results obtained are presented in FIG. 1A.

c. Presence of Anti-PTX3 Antibodies in IFI+PR3+(MPO−) Patients

Sera from 22 patients presenting with polynuclear autoantibodies (IF+) and anti-PR3 antibodies and from 23 healthy subjects were tested as described above. The threshold value (mean+2sd) is 0.04-11 patient sera were higher than the threshold value. The results obtained are presented in FIG. 1B.

d. Presence of Anti-PTX3 Antibodies in IFI+MPO−PR3− Patients

Sera from 21 patients presenting with polynuclear autoantibodies (IFI+) and 23 healthy subjects were tested as described above. The threshold value (mean+2sd) is 0.3-9 patient sera were higher than the threshold value. The results obtained are presented in FIG. 2.

e. Analysis of Anti-PTX3 Antibody Frequency in ANCA+MPO−PR3− Patients

Investigation of autoantibodies directed against other specificities (bactericidal permeability increasing protein [BPI]), cathepsin G and elastase) was carried out in IFI+MPO− and PR3− patients using a commercial ELISA kit (ANCAprofil, Euroimmun, XX). In parallel, the presence of these anti-PTX3 antibody sera was investigated by ELISA, as described above.

The results obtained are represented in diagrammatic form below. They show that 72% of patients with IFI+ which cannot be confirmed at present by routine commercial kits (allowing detection of autoantibodies directed against the following antigens: MPO₃, PR3, BPI, cathepsin G and elastase) have anti-PTX3 antibodies.

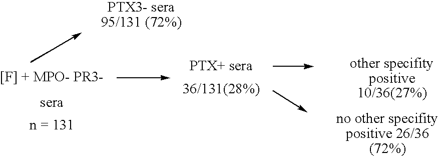

f. Anti-PTX3 in Patients Presenting with Autoantibodies Directed Against Various Autoantigens.

Investigation of anti-PTX3 autoantibodies was investigated by ELISA as described above in the sera of patients presenting with various autoimmune diseases (and in particular systemic diseases). The sera tested were defined on the basis of the presence of:

anti-SSA autoantibodies: these autoantibodies are directed against the nuclear antigen SSA. They are found in Gougerot-Sjögren syndrome (dry syndrome), seperately or associated with another connectivitis.

positive Farr test: which is indicative of the presence of native anti-DNA antibodies. These antibodies are present in 90% of disseminated lupus erythematosis (DLE) cases.

Cyclic citrulline antipeptide autoantibodies (anti-CCP): anti-CCP antibodies are a highly specific diagnostic tool for rheumatoid arthritis.

Anti-Saccharomyces cerevisiae antibodies (ASCA): ASCAs are directed against a structural epitope (Manα-1.2Man) ₙα-1-3Man, n= or >1 and are recognised as being associated with Crohn's disease.

The results obtained are summarised in Table 1.

TABLE 1
Serum autoantibodies (anti-SSA, anti-native DNA, anti-
Saccharomyces cerevisiae [ASCA], anti-CCP) are diluted to
1/400 as described previously and tested in ELISA
in parallel to sera from healthy subjects.

| | | Number of | Frequency of |
|---|---|---|---|
| | | sera | sera |
| Serum | Number of | presenting | presenting |
| characteristics: | sera | anti-PTX3 | anti-PTX3 |
| presence of | tested: | antibodies | antibodies |
| Anti-SSA | 11 | 2 | 2/11 |
| Anti-native ADN | 10 | 4 | 4/10 |
| ASCA | 10 | 3 | 3/10 |
| Anti-CCP | 10 | 1 | 1/10 |

### BIBLIOGRAPHY

Abderrahim-Ferkoune A, Bezy O, Chiellini C, Maffei M, Grimaldi P, Bonino F, Moustaid-Moussa N, Pasqualini F, Mantovani A, Ailhaud G, Amri E Z. Characterization of the long pentraxin PTX3 as a TNFalpha-induced secreted protein of adipose cells. J Lipid Res. 2003, 44:994-1000.
Agrawal A, Volanakis J E. Probing the Clq-binding site on human C-reactive protein by site-directed mutagenesis. J. Immunol. 1994, 152:5404-10.
Alles V V, Bottazzi B, Peri G, Golay J, Introna M, Mantovani A. Inducible expression of PTX3, a new member of the pentraxin family, in human mononuclear phagocytes. Blood. 1994, 84:3483-93.
Bijl M, Horst G, Bijzet J, Bootsma H, Limburg P C, Kallenberg C G. Serum amyloid P component binds to late apoptotic cells and mediates their uptake by monocyte-derived macrophages. Arthritis. Rheum. 2003, 48:248-54.
Bottazzi B, Vouret-Craviari V, Bastone A, De Gioia L, Matteucci C, Peri G, Spreafico F, Pausa M, D'Ettorre C, Gianazza E, Tagliabue A, Salmona M, Tedesco F, Introna M, Mantovani A. Multimer formation and ligand recognition by the long pentraxin PTX3. Similarities and differences with the short pentraxins C-reactive protein and serum amyloid P component. J. Biol. Chem. 1997, 272:32817-23.
Breviario F, dAniello E M, Golay J, Peri G, Bottazzi B, Bairoch A, Saccone S, Marzella R, Predazzi V, Rocchi M, et al. Interleukin-1-inducible genes in endothelial cells. Cloning of a new gene related to C-reactive protein and serum amyloid P component. J Biol. Chem. 1992, 267:22190-7.
Dias A A, Goodman A R, Dos Santos J L, Gomes R N, Altmeyer A, Bozza P T, Horta M F, Vilcek J, Reis L F. TSG-14 transgenic mice have improved survival to endotoxemia and to CLP-induced sepsis. J. Leuk. Biol. 2001, 69:928-36.
Diniz S N, Nomizo R, Cisalpino P S, Teixeira M M, Brown G D, Mantovani A, Gordon S, Reis L F, Dias A A. PTX3 fonction as an opsonin for the dectm-1-dependent internalization of zymosan by macrophages. J Leukoc Biol. 2004 75, 649-56.
Doni A, Peri G, Chieppa M, Allavena P, Pasqualini F, Vago L, Romani L, Garlanda C, Mantovani A. Production of the soluble pattern recognition receptor PTX3 by myeloid, but not plasmacytoid, dendritic cells. Eux J. Immunol. 2003, 33:2886-93.
Garlanda C, Bottazzi B, Bastone A, Mantovani A. Pentraxins at the crossroads Between Imiate Immunity, Inflammation, Matrix Deposition and Female Fertility. Annu Rev Immunol. 2005, 23: 337-366.
Garlanda C, Hirscli E, Bozza S, Salustri A, De Acetis M, Nota R, Maccagno A, Riva F, Bottazzi B, Peri G, Doni A, Vago L, Botto M, De Santis R, Carminati P, Siracusa G, Altruda F, Vecchi A, Romani L, Mantovani A. Non-redundant role of the long pentraxin PTX3 in anti-fungal innate immune response. Nature. 2002, 420:182-6.
Gershov D, Kim S, Brot N, Elkon K B. C-Reactive protein binds to apoptotic cells, protects the cells from assembly of the terminal complement components, and sustains an antiinflammatory innate immune response: implications for systemic autoimmunity. J. Exp. Med. 2000, 192:1353-64.
Goodman A R, Levy D E, Reis L F, Vilcek J. Differential regulation of TSG-14 expression in murine fibroblasts and peritoneal macrophages. J Leukoc Biol. 2000, 67:387-95,
Hind C R, Collins P M, Renn D, Cook R B, Caspi D, Marilyn L Baltz, and Pepys M B, Binding specificity of serum amyloid P component for the pyruvate acetal of galactose. J Exp Med. 1984 Apr. 1; 159(4):1058-69.
Janeway C A Jr, Medzhitov R., Innate immune recognition. Annu Rev Immunol. 2002; 20:197-216. Epub 2001 Oct. 4. Review.
Lee G W, Lee T H, Vilcek J. TSG-14, a tumor necrosis factor- and IL-1-inducible protein. is a novel member of the pentaxin family of acute phase proteins. J. Immunol. 1993; 150:1804-12.
Matsushita M, Fujita T. The role of ficolins in innate immunity. Immunobiology. 2002, 205:490-7. Nauta A J, Bottazzi B, Mantovani A, Salvatori G, Kishore U, Schwaeble W J, Gingras A R, Tzirna S, Vivanco F, Egido J, Tijsma O, Hack E C, Daha M R, Roos A. Biochemical and functional characterization of the interaction between pentraxin 3 and Clq. Eur J. Immunol. 2003, 33:465-73.
Medzhitov R, Janeway C Jr., Innate immunity, N Engl J. Med. 2000 Aug. 3; 343(5):338-44. Review.
Nauta A J, de Haij S, Bottazzi B, Mantovani A, Bornas M C, Aten J, Rastaldi M P, Daha M R, van Kooten C, Roos A. Human renal epithelial cells produce the long pentraxin PTX3. Kidney Int. 2005, 67:543-53.
Rovere P, Peri G, Fazzini F, Bottazzi B, Doni A, Bondanza A, Zimmermann V S, Garlanda C, Fascio U, Sabbadini M G, Rugarli C, Mantovani A, Manfredi A A. The long pentraxin PTX3 binds to apoptotic cells and regulates their clearance by antigen-presenting dendritic cells. Blood. 2000, 96:4300-6.
Van de Wetering J K, van Golde L M, Batenburg J J. Collectins: players of the innate immune system. Eux. J. Biochem. 2004, 271:1229-49.
Vouret-Craviari V, Matteucci C, Peri G, Poli G, Introna M, Mantovani A. Expression of a long pentraxin, PTX3, by monocytes exposed to the mycobacterial cell wall component lipoarabinomannan. Infect Immun. 1997, 65:1345-50.

1. A method for in vitro diagnosis of an autoimmune immune response in a subject by detection in a biological fluid of said subject of antibodies directed against the pentraxin 3 antigen (PTX3) wherein a presence of antibodies directed against the PTX3 antigen (anti-PTX3 antibodies) is determined in the serum of said subject and existence of an autoimmune immune response in the subject is concluded on this basis.

2. The method according to claim 1, wherein a quantity of antibodies directed against pentraxin 3 (PTX3) is determined in a biological fluid of the subject and the existence of an autoimmune immune response in the subject is established through comparison with the quantity of antibodies directed against reference PTX3 antigen.

3. The method according to claim 1, wherein the presence and/or quantity of antibodies directed against PTX3 is determined by detection of binding between the PTX3 antigen and the anti-PTX3 antibody.

4. The method according to claim 3, wherein detection of binding between the PTX3 antigen and the anti-PTX3 antibody is carried out by immobilisation of the PTX3 antigen on a solid support, by precipitation reactions in liquid media and/or immunoprecipitation and/or by a gel precipitation reaction.

5. The method according to claim 4, wherein the solid support for immobilisation of the PTX3 antigen is of plastic or polypropylene type for ELISA or RIA assays, of membrane type for Western blot or Dot blot assays, of beads type or of foam type.

6. The method according to claim 5, wherein the presence and/or quantity of anti-PTX3 antibodies is determined using a chromogenic substrate, chemoluminescence, fluorescence or radio-labelling.

7. The method according to claim 5, wherein for quantification of PTX3 antibodies, the method comprises an ELISA or RIA assay and to detect higher quantities of anti-PTX3 antibodies compared to the quantity of reference anti-PTX3 antibodies, the method comprises a Western blot or Dot blot type assay.

8. The method according to claim 1, wherein the quantity of reference anti-PTX3 antibodies is the quantity of anti-PTX3 antibodies obtained from the serum of a healthy subject, from a set of serum from healthy subjects or defined in an arbitrary manner by means which allows reproduction of the mean value obtained with a pool from healthy subjects.

9. The method according to claim 4, wherein the gel precipitation reaction is chosen from radial immunodiffusion, Ouchterlony double immunodiffusion, immunoelectrophoresis and fused rocket electrophoresis type reactions.

10. The method according to claim 1, wherein the biological fluid is serum.

11. The method according to claim 1, wherein it is an ELISA assay and comprises the following steps:
a) incubating the serum of said subject with PTX3 antigens fixed on a solid support,
b) washing the serum antibodies not fixed to PTX3 antigens of the solid support,
c) adding anti-immunoglobulin antibodies coupled to a marker, said anti-immunoglobulin antibodies being capable of recognising serum antibodies,
d) washing the anti-immunoglobulin antibodies not fixed to the solid support, and
e) detecting and/or quantifying the marker bound to the solid support and correlating it to the presence and/or quantity of serum antibodies.

12. The method according to claim 4, wherein the PTX3 antigens are chosen from whole PTX3 of human, animal or synthetic origin, one or more PTX3 fragments of human, animal or synthetic origin, and PTX3 homologue molecules from the pentraxins family and/or presenting substantial homology with the primary, secondary or tertiary sequences.

13. The method according to claim 12, wherein the PTX3 antigens are obtained from a prokaryotic recombinant system, a eukaryotic recombinant system, from purification using human or animal biological cells, tissues or fluids, or by chemical synthesis.

14. The method according to claim 11, wherein the anti-immunoglobulin antibodies are selected from the group consisting of anti-immunoglobulin G, anti-immunoglobulin A, anti-immunoglobulin M, anti-immunoglobulin D, and anti-immunoglobulin E.

15. The method according to claim 11, wherein the solid support comprises microbeads or microtitre plates.

16. The method according to claim 11, wherein the marker is selected from the group consisting of fluorescent, chemoluminescent, enzymatic and radioactive markers.

17. The method according to claim 16, wherein the marker is enzymatic and the corresponding enzymes and soluble substrates are selected from the group consisting of:
Alkaline phosphatase and soluble substrate 4-NitroPhenyl Phosphate (PNPP)
Peroxidase and soluble substrate orthophenylene diamine (OPD)
β-galactosidase and soluble substrate 2-nitrophenyl β-galactoside (ONPG)
Glucose 6-phosphate dehydrogenase and soluble substrate glucose-6-phosphate (G6P) and
Biotin and soluble substrate streptavidin coupled to peroxidase and substrate ABTS or OPD of peroxidase.

18. The method according to claim 1, wherein the subject is a mammal.

19. The method according to claim 18, wherein the subject is a human being.

20. The method according to claim 19, wherein the antibodies are anti-human immunoglobulin antibodies.

21. The method according to claim 1, wherein the diagnosis of an autoimmune immune response is associated with the existence or prediction of an autoimmune disease in the subject.

22. The method according to claim 21, wherein the autoimmune disease is selected from the group consisting of Gougerot-Sjögren syndrome, type 1 diabetes, monoclonal gammapathy, Wegener's granulomatosis, disseminated lupus erythematosus, atheromatous disease, Crohn's disease, Horton's disease, Reiter's disease (conjunctivo-uretro-synovial syndrome), rheumatoid arthritis, haemorrhagic recto-colitis, psoriatic rheumatism, sarcoidosis, sclerodermy, multiple sclerosis and autoimmune bullous dermatoses, Basedow's disease (hyperthyroidism), Hashimoto's chronic thyroiditis (hypothyroidism), Goodpasture's syndrome, pemphigus, myasthenia, insulin resistant diabetes, autoimmune haemolytic anaemia, autoimmune thrombocytopenic purpura, polymyositis and dermatomyositis, Biermer's anaemia, glomerulonephritis, certain sterile diseases, periarteritis nodosum and Churg-Strauss syndrome, Still's disease, atrophying polychondritis, Behçet's disease and spondylarthritis.

23. The method according to claim 1 also comprising detection and/or quantification in the serum of said subject of antibodies directed against the myeloperoxidase antigen (MPO) and/or proteinase 3 antigen (PR3) and/or elastase and/or BPI and/or cathepsin G and/or nuclear antigens.

24. The method according to claim 1, wherein the diagnosis of an autoimmune immune response is associated with a pathology comprising tissue damage in the subject due to necrosis.

25. A diagnostic kit for detection and/or quantification in a biological fluid of antibodies directed against the PTX3 antigen comprising:
a) a solid support, wherein the PTX3 antigen is fixed to the solid support.

26. (canceled)

27. The diagnostic kit according to claim 25, further comprising:
b) a solution containing anti-immunoglobulin antibodies conjugated to a marker.

28. The diagnostic kit according to claim 27, wherein the anti-immunoglobulin antibodies are selected from the group consisting of anti-immunoglobulin G, anti-immunoglobulin A, anti-immunoglobulin M, anti-immunoglobulin D, and anti-immunoglobulin E.

29. The diagnostic kit according to claim 25, further comprising:
c) a washing solution.

30. The diagnostic kit according to claim 27, wherein the solid support is an ELISA plate and the marker is an enzyme.

31. The diagnostic kit according to claim 30, further comprising:
d) a solution containing the soluble substrate corresponding to the enzyme.

32. (canceled)

33. (canceled)

34. A method for identifying, prior to the appearance of clinical symptoms, subjects who run the risk of developing an autoimmune disease, comprising performing the method according to claim 1.

35. A method for monitoring the evolution of an autoimmune immune response, predicting progression of the disease and/or monitoring the efficacy of treatment, comprising performing the method according to claim 1.
